(19) United States
(12) Patent Application Publication
(10) Pub. No.: US 2014/0357963 A1
(43) Pub. Date: Dec. 4, 2014

(54) PORTABLE ELECTRONIC APPARATUS

(71) Applicant: EMINENT ELECTRONIC TECHNOLOGY CORP. LTD., Hsinchu (TW)

(72) Inventors: TOM CHANG, Taipei City (TW); Kao-Pin Wu, New Taipei City (TW); Chih-Jen Fang, Tainan City (TW); Fang-Yu Liu, Hsinchu County (TW); Cheng-Ta Chuang, New Taipei City (TW); Cheng-Te Chan, New Taipei City (TW); De-Cheng Pan, Changhua County (TW)

(73) Assignee: EMINENT ELECTRONIC TECHNOLOGY CORP. LTD., Hsinchu (TW)

(21) Appl. No.: 14/290,996

(22) Filed: May 30, 2014

Related U.S. Application Data
(60) Provisional application No. 61/829,265, filed on May 31, 2013.

(30) Foreign Application Priority Data
May 13, 2014 (TW) 103116878

(51) Int. Cl. A61B 5/00; A61B 5/02; A61B 5/0205; A61B 5/1455

(52) U.S. Cl. CPC A61B 5/6898; A61B 5/14552; A61B 5/4845; A61B 5/02007; A61B 5/0205; A61B 5/082
USPC 600/301; 600/323; 600/532; 600/479; 600/324

(57) ABSTRACT

A portable electronic device for providing a plurality of operation modes, emits light to an object to be sensed via a light source and senses reflected light from the object to be sensed via an optical sensor to generate a sensing signal. Based on this sensing signal, various functions can be performed, for example, remote control, proximity sensing, gesture sensing, hear rate sensing, blood oxygen saturation sensing, exhaled gas alcohol concentration sensing. The portable electronic device provided by the present invention integrate various functions therein and can measure various kinds of physiological parameters, thus is more convenient and helpful for many users.

### CROSS REFERENCE TO RELATED APPLICATIONS

This application claims the benefit of U.S. Provisional Application No. 61/829,265, filed on May 31, 2013, the contents of which are incorporated herein by reference.

### BACKGROUND OF THE INVENTION

1. Field of the Invention

The present invention relates to a portable electronic apparatus, and particularly to a portable electronic apparatus which can perform various functions.

2. Description of the Prior Art

Portable electronic apparatuses, such as smart phones or tablets, are very popular devices, which can support many application programs to meet various user requirements. As well as the standard telephone functions, these application programs can provide other functions for a user, such as: heart rate sensing, blood oxygen saturation sensing, alcohol concentration sensing, and a remote control function. It would be desirable for the remote control to be applied to control different appliances such as a television, a stereo cabinet, or an air conditioner. If all the above-mentioned functions can be integrated in a single portable electronic apparatus, the convenience to a user could be greatly increased.

### SUMMARY OF THE INVENTION

Therefore, one objective of the present invention is to provide a portable electronic apparatus, which can use the same or a same group of devices to perform different functions.

Another objective of the present invention is to provide a portable electronic apparatus which can sense physiological parameters of human body anytime and anywhere.

One embodiment of the present invention discloses a portable electronic apparatus, which comprises: a first switching unit, for switching the portable electronic apparatus to operate in a remote control mode; a first internal light source; a remote control signal emitter; an optical sensor, comprising at least one optical sensing unit, for sensing reflected light generated by the first internal light source to an object to generate a gesture sensing signal in the remote control mode; and a control unit, coupled to the optical sensor and the remote control signal emitter, wherein, in the remote control mode, the control unit determines a gesture according to the gesture sensing signal, and controls the remote control signal emitter to emit a first remote control signal corresponding to the gesture for wirelessly controlling a target electronic apparatus.

Another embodiment of the present invention discloses a portable electronic apparatus, which comprises: a first switching unit, for switching the portable electronic apparatus to operate in a first sensing mode to sense a heart rate of a user; a first internal light source, for emitting light to an object to be sensed; an optical sensor, comprising at least one optical sensing unit, for sensing reflected light generated from the object to be sensed; and a control unit, coupled to the optical sensor, for determining if any object approaches the optical sensor according to an output of the optical sensor in a proximity sensing mode, and for determining a heart rate of a user according to the output of the optical sensor in the first sensing mode.

Many functions are therefore integrated in a single portable electronic apparatus. Accordingly, the user only needs to carry one electronic apparatus which increases the convenience for the user. The portable electronic apparatus of the present invention can provide many functions which can sense physiological parameters and can be easily switched, which provides further advantage for the user.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram illustrating a mobile phone according to one embodiment of the present invention.

FIG. 2 is a block diagram illustrating an internal structure for a mobile phone according to one embodiment of the present invention.

FIG. 3 is a schematic diagram illustrating how to use a mobile phone to control a target electronic apparatus according to one embodiment of the present invention.

FIG. 4 is a schematic diagram illustrating an operation of a mobile phone receiving a second remote control signal from other remote control apparatuses according to one embodiment of the present invention.

FIG. 5(a), FIG. 5(b) and FIG. 5(c) are schematic diagrams illustrating an operation of an optical sensor in a mobile phone sensing reflected light from a finger according to one embodiment of the present invention.

FIG. 6(a) and FIG. 6(b) are schematic diagrams illustrating a watch according to an embodiment of the present invention.

FIG. 7 is a block diagram illustrating an example of the optical sensor in the mobile phone according to an embodiment of the present invention.

### DETAILED DESCRIPTION

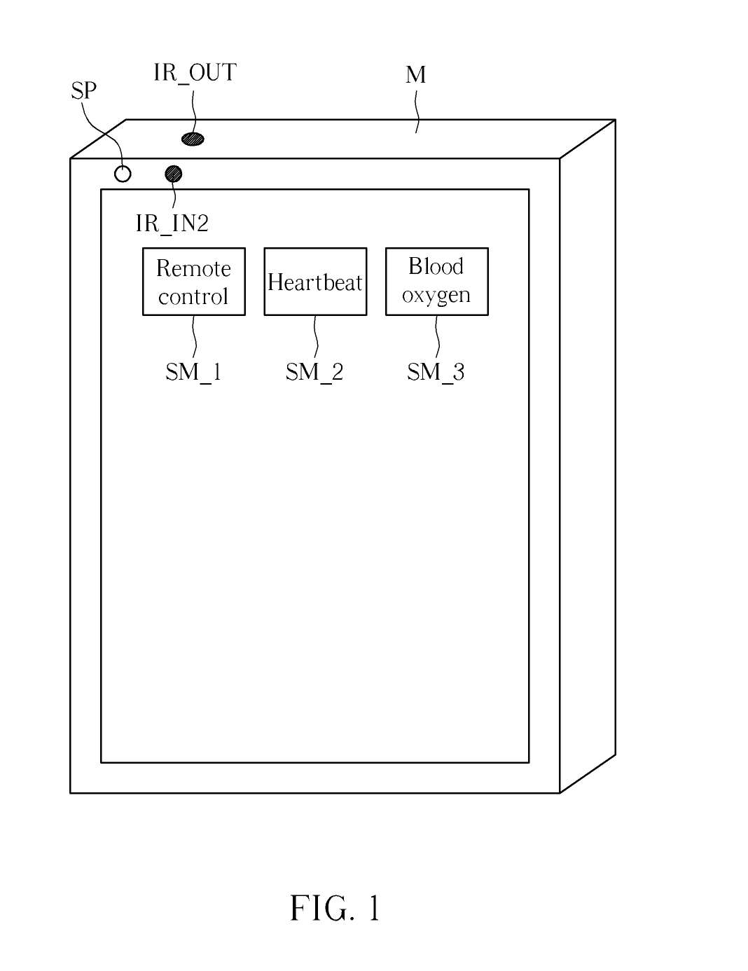

Different figures are provided to explain the content of the present invention. Note that a mobile phone and a finger of a user are applied as examples; however, the mobile phone can be replaced by other kinds of portable electronic apparatus such as a tablet PC or a wearable device. Additionally, the finger can be replaced by other objects such as a hand or palm of the user for some functions. FIG. 1 is a schematic diagram illustrating a mobile phone according to one embodiment of the present invention. The mobile phone M comprises a sensing region SP (e.g. an opening), an internal light source IR_IN2 and an external light source IR_OUT. If an object (e.g. a user's finger) approaches or touches the sensing region SP, another light source IR_IN1 (not illustrated in FIG. 1) of the mobile phone M or the internal light source IR_IN2 can emit light to the user's finger to generate reflected light. The optical sensor (or image sensor), which is not illustrated in FIG. 1, in the mobile phone M senses the reflected light and generates a sensing signal. The external light source IR_OUT emits the light signal to other electronic apparatuses. The internal light source IR_IN2, the external light source IR_OUT and another internal light source in the mobile phone M all generate red light or infrared light, but this is not limited thereto. In other embodiments, the mobile phone M may comprise only an internal light source, such as the internal light source IR_IN2, the internal light source IR_IN1 or other light sources. Various functions can be performed via the devices depicted in FIG. 1. The mobile phone M can sense environmental light in an environmental light sensing mode, sense if an object approaches a surface of the mobile phone in a proximity sensing mode, control a target electronic apparatus (e.g. a television) in a remote control mode, sense a gesture performed near a surface of the mobile phone in a gesture sensing mode, or sense a gas alcohol concentration level in the gas exhaled by an user in an alcohol sensing mode. The mobile phone M can also sense a heart rate, a blood oxygen saturation level, an angiosclerosis parameter, or an angiemphraxis parameter of a user. Note the sensing region SP can be integrated with other devices (e.g. a touch screen) as well as being independent from other devices as depicted in FIG. 1. The only requirement for the sensing region is that light can pass through.

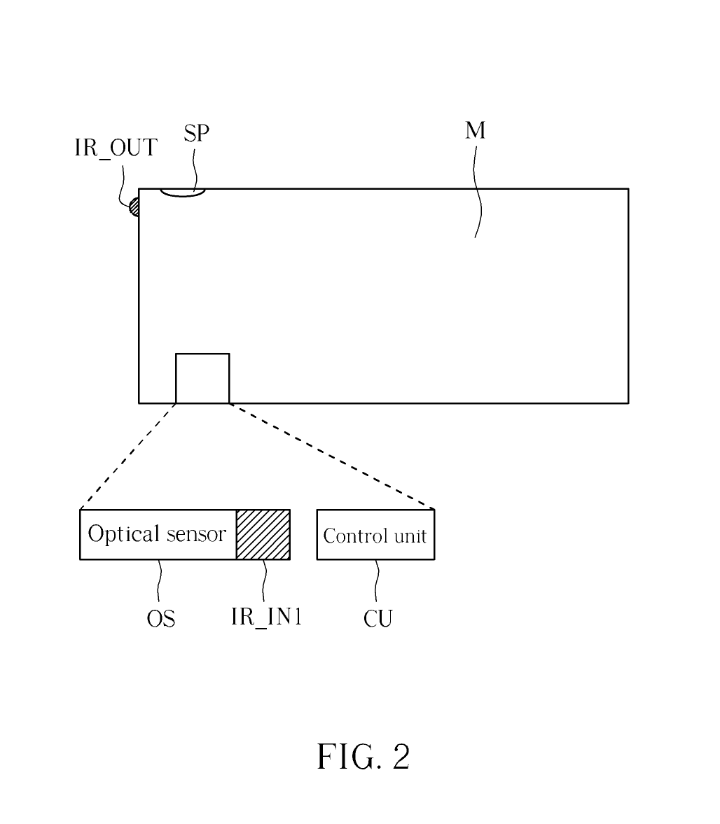

FIG. 2 is a block diagram illustrating the structure of a mobile phone according to one embodiment of the present invention. The mobile phone M comprises an internal light source IR_IN1, an external light source IR_OUT, a control unit CU and an optical sensor OS. Note that the internal light source IR_IN1 is different from IR_IN2 depicted in FIG. 1. IR_IN2 is not illustrated in FIG. 2 due to the figure angle. The optical sensor OS comprises at least one light sensing unit (e.g. a single pixel, or a pixel array), and generates a first optical sensing signal according to reflected light generated by emitting light via the internal light source IR_IN1 to a finger positioned above the optical sensor, or further generates a second optical sensing signal according to reflected light generated by emitting light via the internal light source IR_IN2 to the finger positioned above the optical sensor. In one embodiment, the sensing region SP is an opening, and the internal light source IR_IN1 can emit light to a finger positioned above the sensing region SP via the opening. The optical sensor OS receives reflected light from the finger to generate a first optical sensing signal.

The control unit CU can perform the above-mentioned functions according to the first optical sensing signal, or according to the first optical sensing signal and the second optical sensing signal. The mobile phone M comprise at least one switching unit to switch the operation mode of the mobile phone M for performing different function. The switching unit can be an application program icon displayed on a display of the mobile phone. Application programs can be installed on the mobile phone M, such that the mobile phone M can display different application program icons SM_1, SM_2 and SM_3. If any icon is triggered (e.g. by selecting an icon via a finger), the mobile phone M would enter a corresponding function mode. Different icons indicate different switching units and correspond to different function modes. The switching unit is not limited to be implemented by software. For example, at least one hardware switch can be provided to the mobile phone M, enabling the user to easily switch to a desired function. In one embodiment, the optical sensor OS and the internal light source IR_IN1 are integrated into a single IC (integrated circuit) package.

The above-mentioned functions will be described below. As mentioned above, the mobile phone M can sense environmental light, sense if an object approaches the mobile phone's surface, act as a remote controller to control a target electronic apparatus such as a television, sense a gesture performed near the mobile phone's surface, and sense a gas alcohol concentration level of gas exhaled by a user. When sensing the environmental light, the optical sensor OS senses the intensity of the environmental light. If the display for the mobile phone M comprises a backlight module, the control unit CU may controls the backlight module according to a result that the optical sensor senses the environmental light in order to adjust the brightness of the display. In one embodiment, the mobile phone M enters the environmental light sensing mode while the display is awake. The operation for waking the display can be performed via a button. Therefore, it can be regarded that the mobile phone M enters the environmental sensing mode via a button. Many methods can be applied to sense if an object approaches the mobile phone's surface, one of which is emitting light from the light source (IR_IN1 or IR_IN2). If the object approaches the surface, the light emitted by the light source is reflected by the object, and the optical sensor OS receives the reflected light to generate a sensing signal and outputs the sensing signal. The closer the object is to the surface, the larger the intensity of the reflected light. The control unit CU determines if the object approaches according to the output of the optical sensor OS. In one embodiment, the mobile phone can automatically enter the proximity sensing mode if a call is received, and turns off touch sensing for the mobile phone if the mobile phone senses a large object such as a human face approaches. Many well-known techniques can also sense the environmental light to control the backlight module, but are omitted here for brevity.

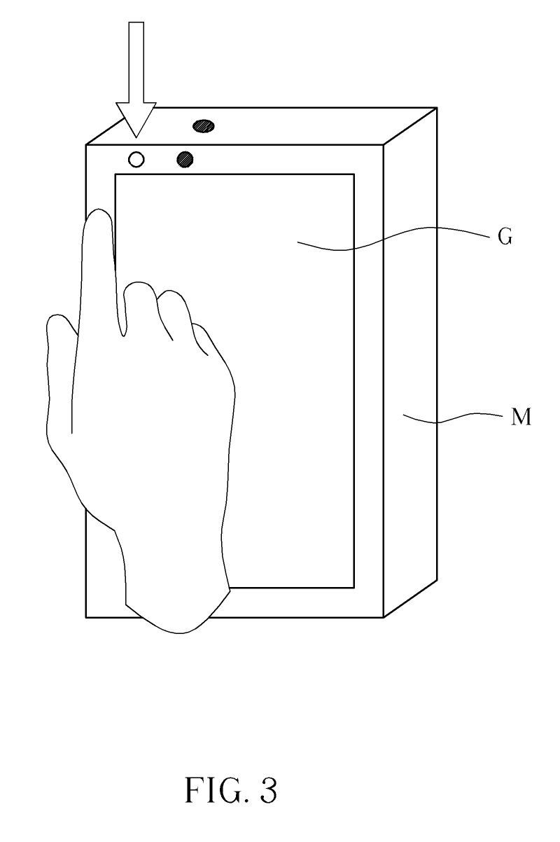

FIG. 3 is a schematic diagram illustrating a mobile phone in a remote control mode controls a target electronic apparatus (a television in this example) according to an embodiment of the present invention. In such embodiment, the mobile phone can perform the function of a remote control. The user may perform a gesture G above the optical sensor of the mobile phone M after the mobile phone M is switched to a remote control mode via a switching unit (e.g. the icon SM_1 in FIG. 1). The gesture G enables the mobile phone M to generate a first remote control signal OP to wirelessly control the control target electronic apparatus T. The optical sensor OS in FIG. 2 senses reflected light generated via emitting light by the internal light source IR_IN1 to a finger to generate a sensing signal (in this mode, the sensing signal is a gesture sensing signal). The control unit CU controls a remote control signal emitter (e.g. the external light source IR_out) to emit a first remote control signal OP corresponding to the sensing signal (i.e. corresponding to the user's gesture) to wirelessly control the target electronic apparatus T in the remote control mode. In one embodiment, the user may perform a gesture by waving her hand to the left or to the right above the mobile phone M, to select a TV channel or to control a TV volume.

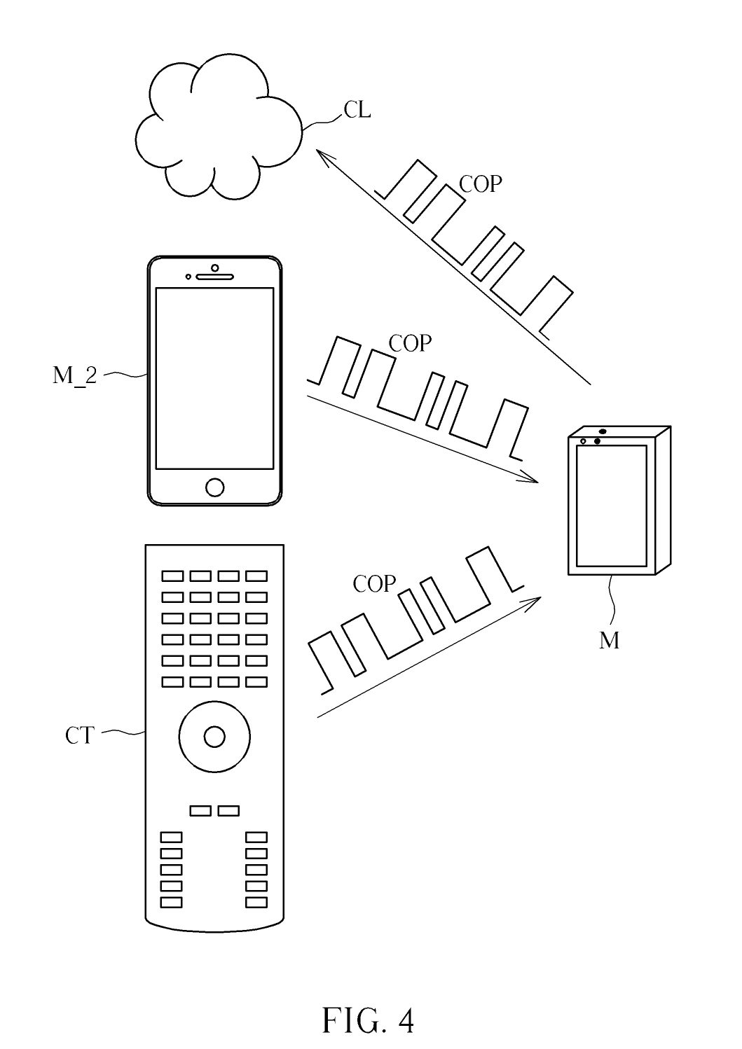

In one embodiment, the mobile phone M enters a learning mode via at least one switching unit. The optical sensor in FIG. 2 receives a second remote control signal emitted from another external remote control device, and the control unit CU stores the second remote control signal to a storage apparatus. Via the learning mode, the mobile phone M can learn the control signal from other remote controllers, such that the mobile phone M can control different electronic apparatuses, or the same kind, but different brand, of electronic apparatus, such as a television, a stereo cabinet, or an air conditioner. The mobile phone M can integrate functions of different remote controllers in this way. After learning the remote control signals from other remote controllers, the control unit CU can control the external light source IR_OUT to emit the first remote control signal to control other electronic apparatuses according to stored second remote control signals. FIG. 4 is a schematic diagram illustrating the operation of a mobile phone receiving a second control signal from other remote control apparatuses according to one embodiment of the present invention. As shown in FIG. 4, in the learning mode, the mobile phone M can receive the second remote control signal COP via another mobile phone M_2 or another remote controller CT, and the received second remote control signal COP can be stored to a storage apparatus in the mobile phone M or in a cloud system CL. In one embodiment, the above-mentioned first remote control signal and the second remote control signal are both optical patterns.

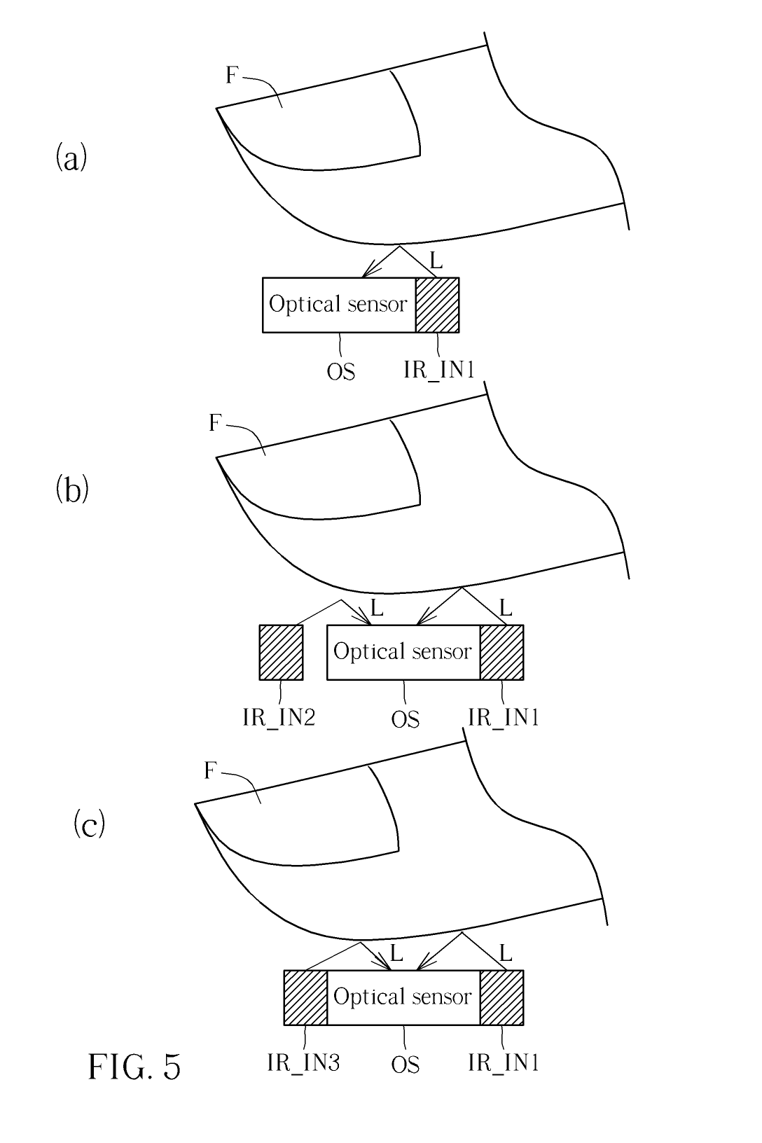

FIG. 5(a), FIG. 5(b) and FIG. 5(c) are schematic diagrams illustrating the operation of an optical sensor in a mobile phone sensing reflected light from a finger according to one embodiment of the present invention. FIG. 5(a) illustrates the mobile phone M comprising a single light source IR_IN1. FIG. 5(b) illustrates the situation where the mobile phone M further comprises another internal light source IR_IN2, wherein the optical sensor OS is integrated with the internal light source IR_IN1, but the light source IR_IN2 is independent from the optical sensor OS. FIG. 5(c) illustrates the mobile phone M comprising yet another internal light source IR_IN3, wherein the optical sensor OS is integrated with both the internal light sources IR_IN1 and IR_IN3; for example, integrated in a signal IC package.

As shown in FIG. 5(a), the internal light source IR_IN1 emits the light L to the finger F to generate the reflected light, and the optical sensor OS generates an optical signal according to the reflected light. In FIG. 5(b) and FIG. 5(c), the optical sensor OS generates an optical signal according to the reflected light generated via emitting light by the internal light source IR_IN1 to the finger F, and generates another optical signal according to the reflected light generated via emitting light by the internal light source IR_IN2 or IR_IN3 to the finger F. In one embodiment, the internal light sources IR_IN1 and IR_IN2 (or IR_IN3) alternatively emit light, and the optical sensor OS alternatively receives the reflected light generated via emitting light by the internal light source IR_IN1 to the finger F, and receives the reflected light generated via emitting light by the internal light source IR_IN2 (or IR_IN3) to the finger F, to generate an output. The control unit determines the movement for the object according to the output of the optical sensor.

The structures in FIG. 5(a), FIG. 5(b) and FIG. 5(c) can be applied to perform the gesture sensing operation in the embodiment shown in FIG. 3. In the structure of FIG. 5(a), the internal light source IR_IN1 continuously emits light to the finger F to generate reflected light while the finger F is moving. The optical sensor OS comprises a plurality of pixels (i.e. a plurality of optical sensing units). The closer the pixel is from the finger, the larger the intensity of light received by the pixel. Therefore, the location or the movement of the finger can be determined according to reflected light information received by each pixel. The reflected light received by each pixel is transferred to the output of the optical sensor OS, and the control unit CU can accordingly determine the movement of the finger. In the structures of FIG. 5(b) and FIG. 5(c), the light sources in the figure alternatively emit light, and one pixel (i.e. one optical sensing unit) is sufficient for the optical sensor to determine the movement of the finger. In other embodiments, more than one pixel can be applied simultaneously. The internal light source IR_IN1 and IR_IN2 (or IR_IN3) continuously emit light to the finger F to generate reflected light while the finger F is moving. Since the two light sources emit light alternatively, the single pixel of the optical sensor OS receives reflected light generated by emitting light to the finger by two light sources in the order in which the light sources emit light. Two reflected light waves with different phases can be acquired according to the order that the light sources emit light and the sensing result of the pixel. For example, the signal sensed by the optical sensor belongs to the first wave if the light source IR_IN1 emits light, and the signal sensed by the optical sensor belongs to the second wave if the light source IR_IN2 (or IR_IN3) emits light. The control unit CU can determine the movement of the finger according to phase relations between these two signals (leading or lagging). Note the gesture sensing operation in FIG. 3 is not limited to the operation corresponding to FIG. 5(a), FIG. 5(b) and FIG. 5(c), and the gesture can be performed by a finger, a palm or a hand of a user. For example, the whole pixel array in the optical sensor OS can be applied to sense the image, and compute the location and movement path for the finger according to the image sensed by the optical sensor OS. If the light sources IR_IN1, IR_IN2 are applied and the optical sensor comprises a single pixel, determining a gesture in a single axis can be performed. For example, the hand moves from left to right or from right to left. If the optical sensor comprises at least three pixels, the determining of gesture in four directions: up, down, left, and right, can be performed. If the optical sensor comprises a pixel array (e.g. 8×8), the location and movement path for the finger can be computed according to the images sensed by the pixel array. The gesture is not limited to the above examples. Since such technique can be implemented by various methods and is well known by persons skilled in the art, it is omitted for brevity here.

If an exhaled gas alcohol concentration level needs to be sensed, the mobile phone can be switched to an alcohol sensing mode via another switching unit (e.g. the application program icon displayed on a display of the mobile phone). The structure in FIG. 5(a) can be applied to perform this sensing, but is not limited thereto. The user exhales towards the sensing region, the internal light source IR_IN1 emits light to the gas to generate reflected light, and the optical sensor OS receives the reflected light and generates a corresponding optical signal (i.e. an alcohol sensing signal). The control unit CU compares the optical signal with predetermined data to compute the gas alcohol concentration level. The predetermined data is associated with the output for the optical sensor OS in a condition that the gas contains no alcohol. Such data can be pre-stored in a storage apparatus in the portable electronic apparatus (the mobile phone). In one embodiment, infrared light with a wave length of 850 nm can be applied to sense the exhaled gas alcohol concentration level, but is not limited thereto. Alcohol absorbs infrared light of a particular wave length. The infrared light emitted from the light source will be absorbed by a gas containing alcohol exhaled by a user. In this case, the optical sensor OS outputs a value lower than a value where no infrared light is absorbed by alcohol. Based on these sensing methods, the exhaled gas alcohol concentration level can be acquired via quantitatively analyzing.

The present invention can also be applied to sense physiological parameters such as: heart rate, a blood oxygen saturation level, an angiosclerosis parameter, or a angiemphraxis parameter. Upon performing these sensing, the user only needs her finger to approach or contact the sensing region SP. The sensing result can be transmitted and stored in the cloud system.

The mobile phone can be switched to a blood sensing mode via a switching unit, to perform a heart rate sensing, an angiosclerosis parameter sensing, or an angiemphraxis parameter sensing. The structure in FIG. 5(a) can be applied to these operations. In the human body, blood is pumped to the whole body when the heart contracts and the blood flows back to the heart when the heart relaxes. If the light source IR_IN1 continuously emits light to the finger F, after a period of time the optical sensor will acquire reflected light for different situations such as blood flowing to the finger or leaving the finger, and generate a blood sensing signal. The control unit performs a Fourier transform according to the blood sensing signal to generate a spectrum and acquires the heart rate according to the spectrum. while sensing the angiosclerosis parameter or the angiemphraxis parameter, the control unit CU can process the blood sensing signal via a conventional mechanism to acquire the angiosclerosis parameter or the angiemphraxis parameter. This is to say if the blood sensing signal is processed by different algorithms, different physiological parameters can be acquired. In one embodiment, infrared light with a wave length of 800 nm-1000 nm can be applied to perform these sensing operations. Since such technique can be implemented by various methods and is well known by persons skilled in the art, it is omitted for brevity here. In different embodiments, the above-mentioned three sensing operations can be independent. For example, the heart rate sensing can be independent from other similar sensing operations, so that the mobile phone can be switched to a heart rate sensing mode which only senses the heart rate.

Via another switching unit, the mobile phone can be switched to a blood oxygen sensing mode. The blood oxygen saturation sensing operation can be performed via the structure in FIG. 5(b). Infrared light with different wave lengths (e.g. 840 nm and 940 nm) is emitted by internal light sources IR_IN1 and IR_IN2 to the user's finger. The optical sensor receives reflected light corresponding to these different wave lengths and generates corresponding optical signal (blood oxygen sensing signal), and the control unit computes the blood oxygen saturation level according to the optical signals. Alternatively, the internal light sources IR_IN3 or IR_IN1 in the structures of FIG. 5(c) can be applied to the sensing operation for the blood oxygen saturation level. In one embodiment, the optical sensor comprises only one pixel (i.e. only one optical sensing unit), and the internal light sources IR_IN1 and IR_IN2 alternatively turn on such that the optical sensor can acquire reflected light corresponding to different light sources. In another embodiment, the internal light sources IR_IN1 and IR_IN2 simultaneously turn on and the optical sensor comprises two pixels (i.e. the optical sensing units). One pixel only receives reflected light corresponding to the internal light source IR_IN1 and the other pixel only receives reflected light corresponding to the internal light source IR_IN2, so that the optical sensor can acquire reflected light corresponding to different light sources. Since emitting light with different wave lengths to blood has oxygen or has no oxygen will cause reflected light with different energy the blood oxygen saturation level can be acquired by processing the output of the optical sensor with appropriate algorithm. Since such technique can be implemented by various methods and is well known by persons skilled in the art, it is omitted for brevity here.

FIG. 5(a), FIG. 5(b) and FIG. 5(c) are only exemplary schematic diagrams, and the distance between the finger and the optical sensor maybe different for different functions. For example, a finger (ex. the finger pulp) should be closer to the optical sensor when sensing physiological parameters such as heart rate, blood oxygen, the angiosclerosis parameter, or the angiemphraxis parameter, but should be further from the optical sensor (ex. 2-10 cm) when performing a gesture.

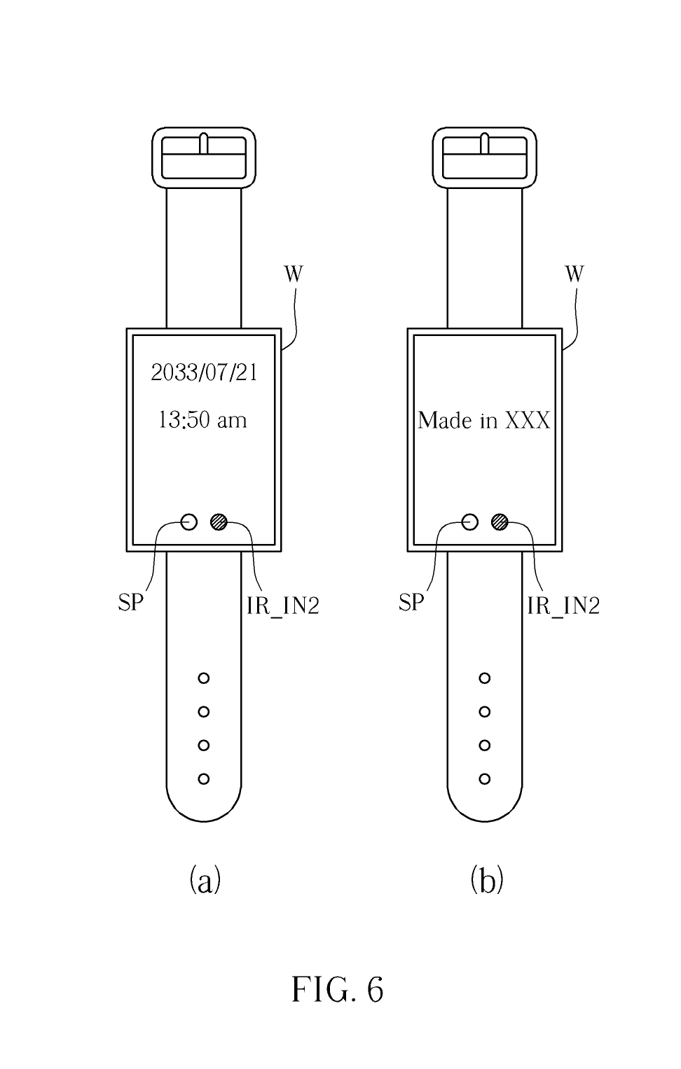

In view of above-mentioned description, the mobile phone provided by the present invention can perform different functions via one or more light sources, an optical sensor and a control unit. These functions can be switched via at least one switching unit. The switching unit can be the application program icons displayed on the display of the mobile phone, or a function list shown by an application program, or a hardware button. As described above, the concept of the present invention is not limited to be applied to a mobile phone, but can also be applied to other portable electronic apparatuses. FIG. 6(a) and FIG. 6(b) are schematic diagrams illustrating a watch according to an embodiment of the present invention. In FIG. 6(a), the sensing region SP and the internal light source IR_IN2 are both provided at a front side of the watch W, where the watch indicates the time. In FIG. 6(b), the sensing region SP and the internal light source IR_IN2 are both provided at a back side of the watch W. Via these embodiments, it should be appreciated that the concept of the present application can be applied to various kinds of portable electronic apparatuses, so that the user is provided with a wide range of selection and greater convenience.

The portable electronic apparatus according to the present invention can perform a gesture sensing and a control operation for a target electronic apparatus, such as the embodiment shown in FIG. 3. The portable electronic apparatus for the embodiment in FIG. 3 can be summarized as: a portable electronic apparatus, comprising: a first switching unit (e.g. SM_1 in FIG. 1), for switching the portable electronic apparatus to operate in a remote control mode; a first internal light source (IR_IN1 in FIG. 2); a remote control signal emitter; an optical sensor, comprising at least one optical sensing unit, for sensing reflected light generated via emitting light by the first internal light source to an object (e.g. a finger or palm of a user) to generate a gesture sensing signal in the remote control mode; and a control unit, coupled to the optical sensor and the remote control signal emitter, wherein, in the remote control mode, the control unit determines a gesture according to the gesture sensing signal and controls the remote control signal emitter to emit a first remote control signal corresponding to the gesture to wirelessly control a target electronic apparatus. The user can control a remote target electronic apparatus (e.g. a television) more conveniently via the gesture.

Based on such embodiment, a portable electronic apparatus control method can be acquired, comprising: (a) generating a first optical sensing signal according to reflected light generated via emitting light by the first internal light source to an object above a sensing region; and (b) controlling a remote control signal emitter to emit a first remote control signal corresponding to the first optical sensing signal to wirelessly control a target electronic apparatus in a first mode.

In another embodiment, the portable electronic apparatus provided by the present invention can sense if any object approaches to thereby sense a heart rate. The portable electronic apparatus can be summarized as: a portable electronic apparatus, comprising: a first switching unit, for controlling the portable electronic apparatus to operate in a first sensing mode to sense a heart rate of a user; a first internal light source, for emitting light to an object to be sensed; an optical sensor, comprising at least one optical sensing unit, for sensing reflected light generated from the object to be sensed; and a control unit, coupled to the optical sensor, for determining if any object approaches the optical sensor according to an output of the optical sensor in a proximity sensing mode, and for determining the heart rate for an user according to an output of the optical sensor in the first sensing mode. The portable electronic apparatus can sense if any object approaches, and thereby enter or leave a sleep mode, to use power more efficiently, and increase the use time of the portable electronic apparatus. Furthermore, the heart rate sensing function can be integrated to the portable electronic apparatus, thus it is helpful for a user who needs to take care his heart rates frequently.

Based on this embodiment, a portable electronic apparatus control method comprises: (a) generating a first optical sensing signal according to reflected light generated via emitting light by the first internal light source to an object above a sensing region; and (b) determining if any object approaches the surface according to an output of the optical sensor in a proximity sensing mode, and determining heart rates for a user according to the first optical sensing signal in a heart rate sensing mode, wherein, in the heart rate sensing mode, the user's finger is above the sensing region.

The scope of the present invention is not limited to the above two embodiments, any combination for above-mentioned functions should fall in the scope of the present invention.

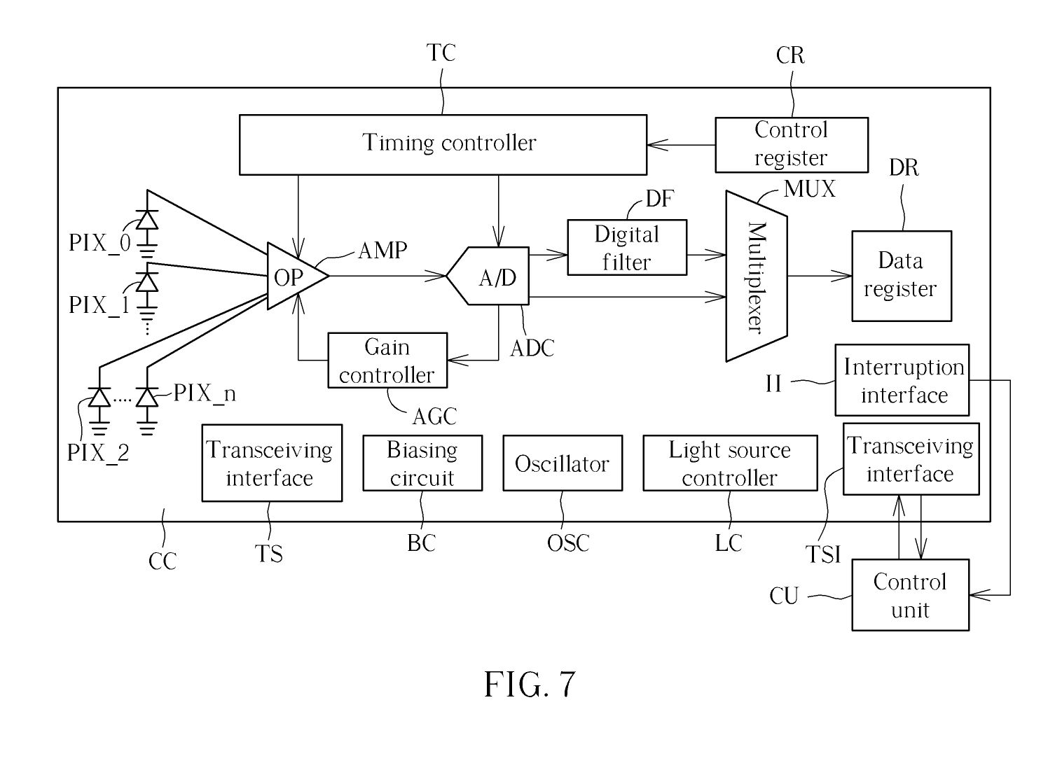

FIG. 7 is a block diagram illustrating an example of the optical sensor in the mobile phone according to the embodiment of the present invention. Note the optical sensor is not limited to have the same structure shown in FIG. 7, and some devices can be omitted according to different function requirements. The optical sensor OS comprises a plurality of optical sensing units PIX_0-PIX_n (e.g. pixel), a timing controller TC, an amplifier AMP, an automatic gain controller AGC, an analog to digital converter ADC, a digital filter DF, a multiplexer MUX, a control register CR, a data register DR, an interruption interface II, a transceiving interface TSI, a light source controller LC, an oscillator OSC, a biasing circuit BC and a temperature sensor TS. The optical sensing units PIX_0-PIX_n sense light to generate the above-mentioned optical sensing signals. The amplifier AMP amplifies the optical sensing signal, and the amplifying ability thereof can be adjusted by the automatic gain controller AGC. The automatic gain controller AGC can adjust the processed optical sensing signal to have a predetermined brightness, via adjusting the gain of the amplifier AMP and the integration time of the optical sensing units PIX_0-PIX_n. The analog to digital converter ADC converts the amplified optical sensing signal to a digital signal. The digital filter filters the noise. The timing controller TC manages the timing for each device in the optical sensor. The temperature sensor TS senses the temperature. The biasing circuit BC is a source to provide bias for the analog circuit. The oscillator OSC is a source for a clock signal. The light source controller LC controls the above-mentioned internal light source or external light source. The control register CR and the data register DR respectively store a command and a sensing result. The transceiving interface TSI receives command/data from the control unit CU or transmits command/data to the control unit CU. The interruption interface II informs the control unit CU a state of the storage space, to determine transceiving of the data.

In view of above-mentioned embodiments, some functions require one light source and one or more optical sensing units, and some functions require at least two light sources and one or more optical sensing unit. By using two light sources with the optical sensor in FIG. 7 which comprising a plurality of optical sensing units, all the above-mentioned functions can be implemented. This means the present invention can provide various functions via a set of hardware devices. In different embodiments, the at least one light source and the at least one optical sensing unit are integrated in a single IC package. The arrangement for the light source(s) and the optical sensing unit(s) can be determined corresponding to different requirements. The IC package comprising the light source and the optical sensing unit generally comprises a glass layer provided on the surface thereof. While performing heart rate and blood oxygen sensing, the user places her finger on this glass layer. The infrared light of the internal light source can be emitted through the glass, reflected from the finger, and received by the optical sensing unit via the glass layer.

In view of above-mentioned embodiments, the devices in a portable electronic apparatus can be shared to perform different functions. Accordingly, the user only needs to carry a single electronic apparatus which is far more convenient. The portable electronic apparatus provided by the present invention can also provide many functions to sense physiological parameters, wherein the function are easily switched.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A portable electronic apparatus, comprising:
a first switching unit, for switching the portable electronic apparatus to operate in a remote control mode;
a first internal light source;
a remote control signal emitter;
an optical sensor, comprising at least one optical sensing unit, for sensing reflected light generated via emitting light by the first internal light source to an object to generate a gesture sensing signal in the remote control mode; and
a control unit, coupled to the optical sensor and the remote control signal emitter, wherein, in the remote control mode, the control unit determines a gesture according to the gesture sensing signal and controls the remote control signal emitter to emit a first remote control signal corresponding to the gesture to wirelessly control a target electronic apparatus.

2. The portable electronic apparatus of claim 1, further comprising a second switching unit, for switching the portable electronic apparatus to a learning mode, in the learning mode the optical sensor receives a second remote control signal emitted from a remote control device, and the control unit stores the second remote control signal to a storage apparatus.

3. The portable electronic apparatus of claim 1, wherein the control unit determines if any object approaches the portable electronic apparatus according to an output of the optical sensor in a proximity sensing mode.

4. The portable electronic apparatus of claim 1, wherein the object is a finger of a user, the portable electronic apparatus further comprises a second switching unit for switching the portable electronic apparatus to a blood sensing mode, in the blood sensing mode the optical sensor senses reflected light generated via emitting light by the first internal light source to the finger to generate a blood sensing signal, and the control unit computes a heart rate, an angiosclerosis parameter, or an angiemphraxis parameter for the user according to the blood sensing signal.

5. The portable electronic apparatus of claim 1, further comprising a second internal light source, wherein the first internal light source and the second internal light source alternatively emit light, the optical sensor comprises a optical sensing unit to receive reflected light generated via emitting light by the first internal light source to the finger and to receive reflected light generated via emitting light by the second internal light source to the finger, and the control unit determines movement for the object according to an output of the optical sensor.

6. The portable electronic apparatus of claim 1, wherein the optical sensor comprises a plurality of optical sensing units for receiving reflected light generated via emitting light by the first internal light source to the object, and the control unit determines movement for the object according to an output of the optical sensor.

7. The portable electronic apparatus of claim 1, wherein the object is a finger of a user, and the portable electronic apparatus further comprises:
a second switching unit, for switching the portable electronic apparatus to a blood oxygen sensing mode; and
a second internal light source, for emitting light having a wave length different from light from the first internal light source;
wherein, in the blood oxygen sensing mode, the first internal light source and the second light source emit light to the finger, the optical sensor generates a blood oxygen sensing signal according to the reflected light from the finger, and the control unit computes a blood oxygen saturation level according to the blood oxygen sensing signal.

8. The portable electronic apparatus of claim 1, further comprising a second switching unit, for switching the portable electronic apparatus to an alcohol sensing mode, wherein, in the alcohol sensing mode, the first internal light source emits light to a gas exhaled by the user, the optical sensor generates an alcohol sensing signal according to the reflected light from the gas, and the control unit computes an exhaled gas alcohol concentration level according to the alcohol sensing signal.

9. The portable electronic apparatus of claim 1, further comprising a display with a backlight module, the optical sensor senses environmental brightness for an environment surrounding the portable electronic apparatus to generate an environmental light sensing signal, and the control unit further controls the backlight module according to the environmental light sensing signal.

10. The portable electronic apparatus of claim 1, wherein the first internal light source and the optical sensor are integrated in an IC package.

11. The portable electronic apparatus of claim 1, further comprising a display, and the first switching unit is an application program icon displayed on the display.

12. A portable electronic apparatus, comprising:
a first switching unit, for switching the portable electronic apparatus to operate in a first sensing mode to sense a heart rate for a user;
a first internal light source, for emitting light to an object to be sensed;
an optical sensor, comprising at least one optical sensing unit, for sensing reflected light generated from the object to be sensed; and
a control unit, coupled to the optical sensor, for determining if any object approaches the optical sensor according to output of the optical sensor in a proximity sensing mode, and for determining a heart rate for a user according to an output of the optical sensor in the first sensing mode.

13. The portable electronic apparatus of claim 12, further comprising a second switching unit, for switching the portable electronic apparatus to a learning mode, in the learning mode the optical sensor receives a remote control signal emitted from an external remote control device, and the control unit stores the remote control signal to a storage apparatus.

14. The portable electronic apparatus of claim 12, further comprising a second internal light source, wherein the first internal light source and the second internal light source alternatively emit light, the optical sensor comprises a optical sensing unit to receive reflected light generated via emitting light by the first internal light source to the finger and to receive reflected light generated via emitting light by the second internal light source to the finger, and the control unit determines movement for the object according to an output of the optical sensor.

15. The portable electronic apparatus of claim 12, wherein the optical sensor comprises a plurality of optical sensing units for receiving reflected light generated via emitting light by the first internal light source to the object, and the control unit determines movement for the object according to an output of the optical sensor.

16. The portable electronic apparatus of claim 12, wherein the object is a finger of a user, and the portable electronic apparatus further comprises:
a second switching unit, for switching the portable electronic apparatus to a blood oxygen sensing mode; and
a second internal light source, for emitting light having a wave length different from light from the first internal light source;
wherein, in the blood oxygen sensing mode, the first internal light source and the second light source emit light to the finger, the optical sensor generates a blood oxygen sensing signal according to the reflected light from the finger, and the control unit computes a blood oxygen saturation level according to the blood oxygen sensing signal.

17. The portable electronic apparatus of claim 12, further comprising a second switching unit, for switching the portable electronic apparatus to an alcohol sensing mode, wherein, in the alcohol sensing mode, the first internal light source emits light to gas exhaled by the user, the optical sensor generates an alcohol sensing signal according to the reflected light from the gas, and the control unit computes an exhaled gas alcohol concentration level according to the alcohol sensing signal.

18. The portable electronic apparatus of claim 12, further comprising a display with a backlight module, wherein the optical sensor senses environmental brightness for an environment surrounding the portable electronic apparatus to generate an environmental light sensing signal, and the control unit further controls the backlight module according to the environmental light sensing signal.

19. The portable electronic apparatus of claim 12, further comprising a display, wherein the first switching unit is an application program icon displayed on the display.

20. The portable electronic apparatus of claim 12, further comprising a second switching unit, for switching the portable electronic apparatus to a remote control mode, and comprising a remote control signal emitter coupled to the control unit;
wherein, in the remote control mode, the optical sensor senses reflected light generated via emitting light by the first internal light source to an object to generate a gesture sensing signal; and the control unit determines a gesture according to the gesture sensing signal and controls the remote control signal emitter to emit a first remote control signal corresponding to the gesture to wirelessly control a target electronic apparatus in the remote control mode.
